(19) United States
(12) Patent Application Publication
(10) Pub. No.: US 2012/0083704 A1
(43) Pub. Date: Apr. 5, 2012

(54) METHOD AND DEVICE FOR BLOOD PRESSURE MEASUREMENT

(75) Inventors: Chih-Cheng Feng, Taipei City (TW); James Hung, Hsinchu County (TW); Fu-Jung Hsiao, Taichung County (TW); Kuo-Hung Chan, Taichung City (TW)

(73) Assignee: INDUSTRIAL TECHNOLOGY RESEARCH INSTITUTE, Hsin-Chu (TW)

(21) Appl. No.: 12/956,938

(22) Filed: Nov. 30, 2010

(30) Foreign Application Priority Data
Sep. 30, 2010 (TW) 099133263

(51) Int. Cl. A61B 5/022

(52) U.S. Cl. 600/490

(57) ABSTRACT

A method and device for blood pressure measurement is disclosed. The steps are capturing the pressure oscillating signal detected from the pressure sensor arranged at the cuff, processing the signal appropriately to cooperate with the personal information inputted by subject or without inputting any information, and actively selecting suitable characteristic coefficient accurately determine the systolic blood pressure and the diastolic blood pressure. It is able to adaptively regulate algorithms relative to different ages, BMIs, medical histories, and further accurately determine the blood pressure, so that the probability of the sphygmomanometer's erroneous judgment is decreased, medical resources are saved, and further a personal blood pressure measurement is accurately performed.

### CROSS-REFERENCE TO RELATED APPLICATIONS

This non-provisional application claims priority under 35 U.S.C. §119(a) on Patent Application No(s). 099133263 filed in Taiwan, R.O.C. on Sep. 30, 2010, the entire contents of which are hereby incorporated by reference.

### TECHNICAL FIELD

The present disclosure relates to a technique for measuring blood pressure, and more particularly, to a method and device for blood pressure measurement.

### TECHNICAL BACKGROUND

As illustrated in the automatic blood pressure monitor that is disclosed in U.S. Pat. No. 4,638,810 by Citron, Inc., a fixed coefficient method is developed for locating and registering a measure of the peak amplitudes of the successively encountered blood flow (oscillatory complex) pulses stored in memory while multiplying the registered peak amplitudes respectively with fixed coefficients so as to be used for determining a subject's systolic and diastolic pressures. Nevertheless, it is noted that the use of the aforesaid fixed coefficient method can cause the probability of the sphygmomanometer's erroneous judgment to increase, as can be inferred from the disclosure of U.S. Pat. No. 4,638,810.

Moreover, as illustrated in the electronic blood pressure measuring instrument that is disclosed in EP 0642,760A1 by Osachi Co. Ltd., not only the blood pressures of the subject can be obtained, but also detection of several diseases can be reliably obtained according to a pattern classification performed upon the oscillating pulse wave detected by the electronic blood pressure measuring instrument, as can be inferred from the disclosure of EP 0642,760A1. However, it is also indicated in the disclosure that since the pulse wave relating to the blood pressure is very much subjected to the influence of the test subject's health condition, an accurate blood pressure determination might be very difficult to achieve, but there is no solution provided in the referring disclosure.

In addition, as illustrated in the electronic blood pressure monitor that is disclosed in CN 1449718A by Omron Co., the systolic pressure of a subject can be estimated according to parameters of pulse wave features, cuff pressures and probability density of pressure values during a pressurizing process while allowing an initial release pressure to be defined as the estimated systolic pressure adding a specific pressure value, as can be inferred from the disclosure of CN 1449718A. However, since the pressuring process prior to the obtaining of blood pressure can only last for a short period of time, accurate blood pressure measurement is hard to achieve.

Furthermore, as illustrated in the electronic blood pressure monitor that is disclosed in U.S. Pat. No. 6,719,703 by VSM Medtech Ltd., a signal envelop is processed for obtaining a maximum blood pressure value while calculating a systolic pressure using the following formulas, as can be inferred from the disclosure of U.S. Pat. No. 6,719,703, which are:

$$1. if ( MAP ≦ ( 90 to 110 ) mmHg ) , then {PIP}_{SBP} = 0.5 to 0.66 ;$$

$$else if ( MAP ≧ ( 130 to 150 ) mmHg ) , then {PIP}_{SBP} = 0.3 to 0.46 ;$$

$${PIP}_{SBP} = α - ( \frac{α - β}{B - A} \times ( MAP - A ) ) . \\ 2. {PIP}_{SBP} = A - \frac{B}{1 + C {e}^{( D \times MAP )}} , C = ( \frac{1}{{e}^{( D \times E )}} ) . \\ 3. {PIP}_{SBP} = A \times {MAP}^{3} + B \times {MAP}^{2} + C \times MAP + D .$$

However, although the measurement of the aforesaid electronic blood pressure monitor is not performed by the used of the fixed coefficient method, it didn't take the personal information, including body mass index (BMI), medical history and age, into consideration, but only focus on how to achieve an accurate measurement within a high pressure range.

Therefore, it is in need of a method and device for blood pressure measurement capable of overcoming the foregoing shortcomings.

### TECHNICAL SUMMARY

The object of the present disclosure is to provide a method and device capable of performing a personal blood pressure measurement accurately.

To achieve the above object, the present disclosure provides a device for blood pressure measurement, comprising: a display unit, for displaying options to be selected by a user; an input unit, provided for the user to selectively input a personal health information or to proceed directly to perform a blood pressure measuring process; an inflation unit; a microprocessor; a cuff; an deflation valve; a pressure sensor; a signal processing unit; and a data processing unit; wherein, the microprocessor is enabled to issue a command for directing the inflation unit to inflate the cuff to a specific pressure, and then the cuff pressure is deflacted by the deflaction valve; and during the pressure deflation, the pressure sensor is enabled to register a pressure signal while feeding the pressure signal to the signal processing unit where it is processed into a data conforming to a format recognizable and usable by posterior calculation processes.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a block diagram showing a blood pressure measuring device according to the present disclosure.

FIG. 2 is a flow chart depicting the steps performed in a blood pressure measuring method of the present disclosure.

FIG. 3 is a flow chart depicting the steps for determining characteristic coefficient, systolic pressure and diastolic pressure in the present disclosure.

FIG. 4 is diagram showing the relationship between initial cuff pressure and arterial pressure values, i.e. systolic pressure and diastolic pressure.

FIG. 5 are diagrams showing the distribution of characteristic coefficients with respective to age groups, BMI groups, and patient groups.

FIG. 6 is a diagram showing an oscillating waveform of the present disclosure.

FIG. 7 is a table showing the comparison between the blood pressure measuring device of the present disclosure and other conventional blood pressure measuring devices.

### DESCRIPTION OF THE EXEMPLARY EMBODIMENTS

For your esteemed members of reviewing committee to further understand and recognize the fulfilled functions and structural characteristics of the disclosure, several exemplary embodiments cooperating with detailed description are presented as the follows.

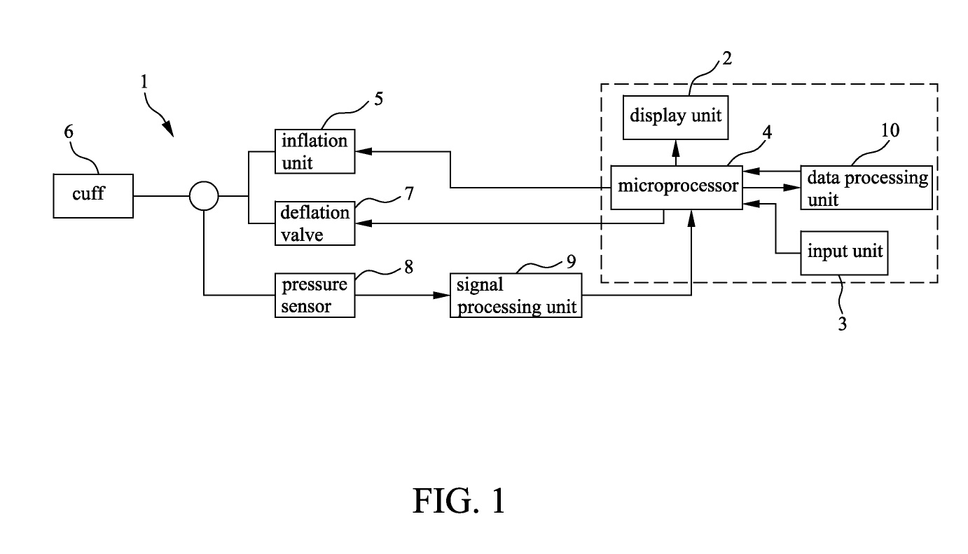

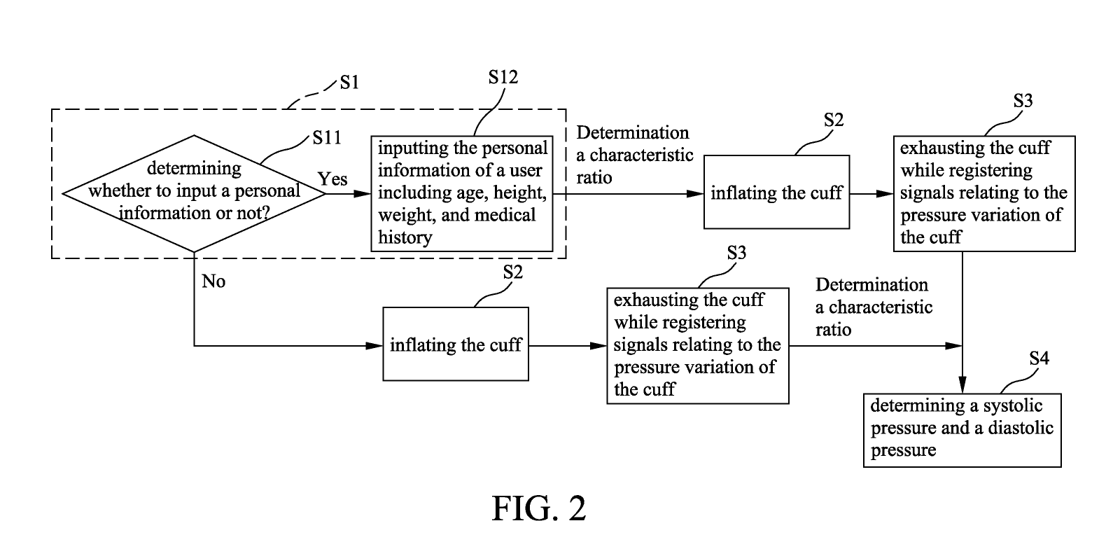

Please refer to FIG. 1 and FIG. 2, which are respectively a block diagram showing a blood pressure measuring device and a flow chart depicting the steps performed in a blood pressure measuring method of the present disclosure.

As shown in FIG. 1, the blood pressure measuring device 1 comprises: a display unit 2, and input unit 3, a microprocessor 4, an inflation unit 5, a cuff 6, an deflation valve 7, a pressure sensor 8, a signal processing unit 9 and a data processing unit 10.

As shown in FIG. 2, the method for blood pressure measurement comprises the steps of:

S1: being composed of two procedures, which are procedure S11 and procedure S12, and at S11, options displayed on the display unit 2 are provided to a user for determining whether to input a personal information or not? If yes, then the flow proceeds to the procedure S12 for inputting the personal information of a user including age, height, weight, and medical history; otherwise, the flow proceeds to step S2;

S2: enabling the microprocessor 4 to issue a command for directing the inflation unit 5 to inflate the cuff 6 to a specific pressure;

S3: stopping the inflation for allowing the cuff 6 to be exhausted by releasing air from the deflation valve 7, and during the exhausting of the cuff 6, enabling the pressure sensor 8 to register a pressure signal while feeding the pressure signal to the signal processing unit 9 where it is processed into a signal envelope conforming to a format recognizable and usable by post-calculation processes, and then transmitting the envelope to the microprocessor 4;

S4: enabling the microprocessor 4 to use the envelope in conjunction with the adaptive characteristic coefficients that are determined by the data processing unit 10 for obtaining a systolic pressure and a diastolic pressure of the user while displaying the two on the display unit 2.

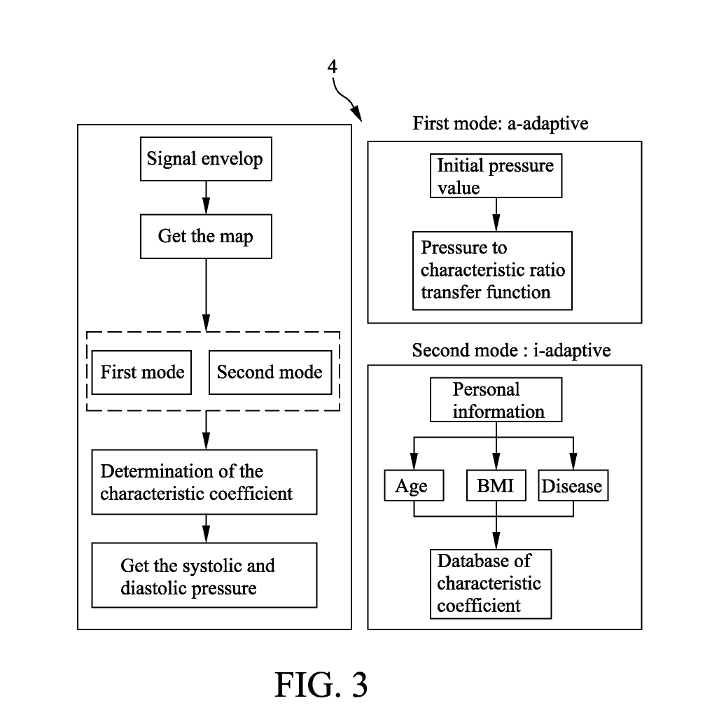

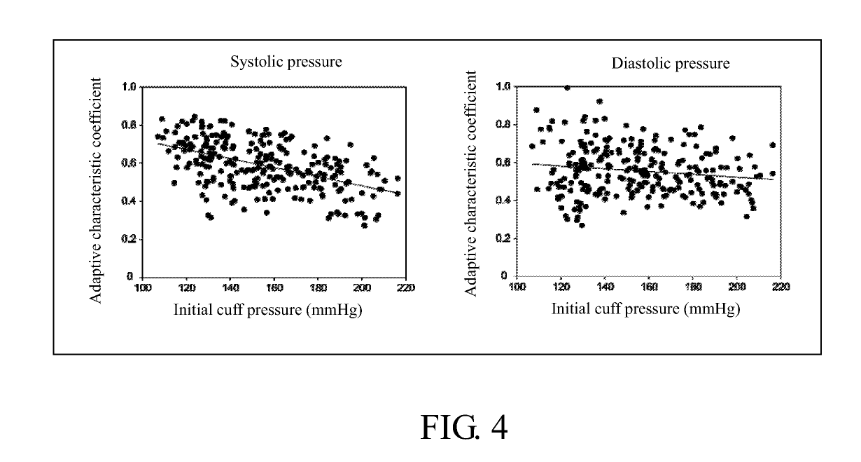

It is noted that the flow chart illustrating the steps for determining the adaptive characteristic coefficients as well as the determination of the systolic pressure and the diastolic pressure thereafter is shown in FIG. 3. As shown in FIG. 3, the flow primarily comprises the steps of: capturing a mean arterial pressure (MAP) value from the envelope of pressure signal; selecting an operation mode from the group consisting of a first mode and a second mode by a user to be used for selecting and determining the adaptive characteristic coefficients corresponding to the selected operation mode. In addition, the first mode is an automatic adaptive (a-adaptive) mode, that is designed for defining the adaptive characteristic coefficient to be selected according to an initial cuff pressure of a cuff 6 mounted on the user so as to be used for the measuring and the determination of the systolic pressure and the diastolic pressure according to the relationship between initial cuff pressure and arterial pressure values, i.e. systolic pressure and diastolic pressure, as shown in FIG. 4.

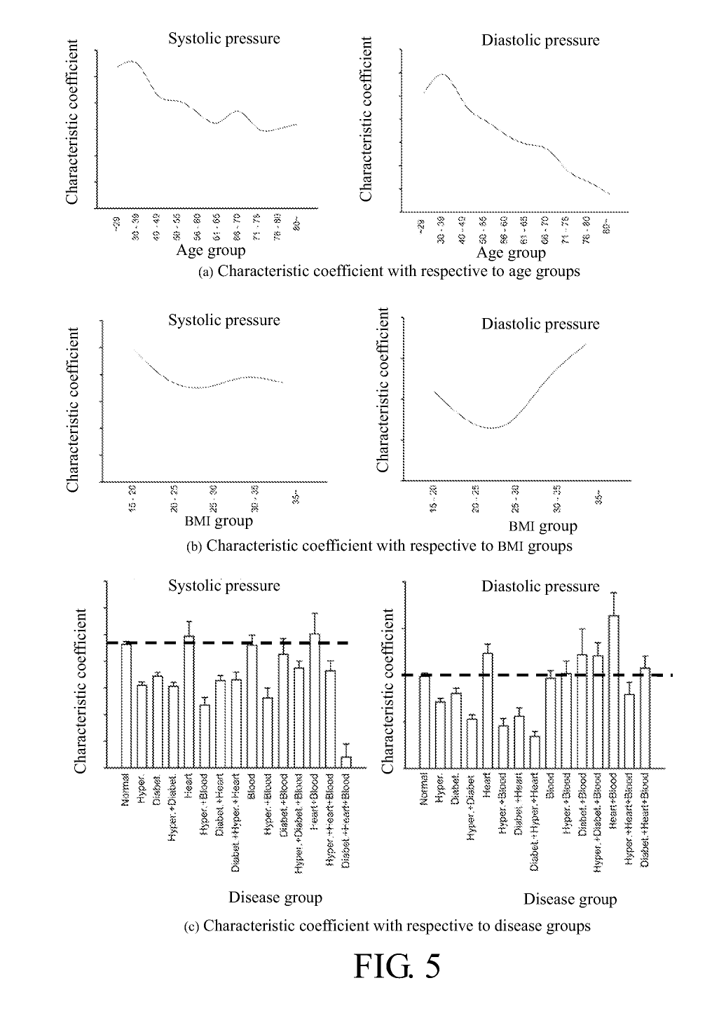

Moreover, the second mode is an intelligent adaptive (i-adaptive) mode, that is designed for defining the adaptive characteristic coefficient to be selected according to a personal information inputted by the user whereas the personal information can include the age, BMI and medical history of the user, in which the medical history further includes patient groups of normal, hypertension, diabetics, heart disease, blood-related disease and the like, while containing the distribution of characteristic coefficients with respective to more than two patient groups, as those shown in FIG. 5.

As shown in FIG. 5, the adaptive characteristic coefficients of the health patient group that are represented as dotted line are different from those of patient groups. Thus, it is required to select the adaptive characteristic coefficient according to the subject's health condition so as to measure the subject's blood pressure accurately.

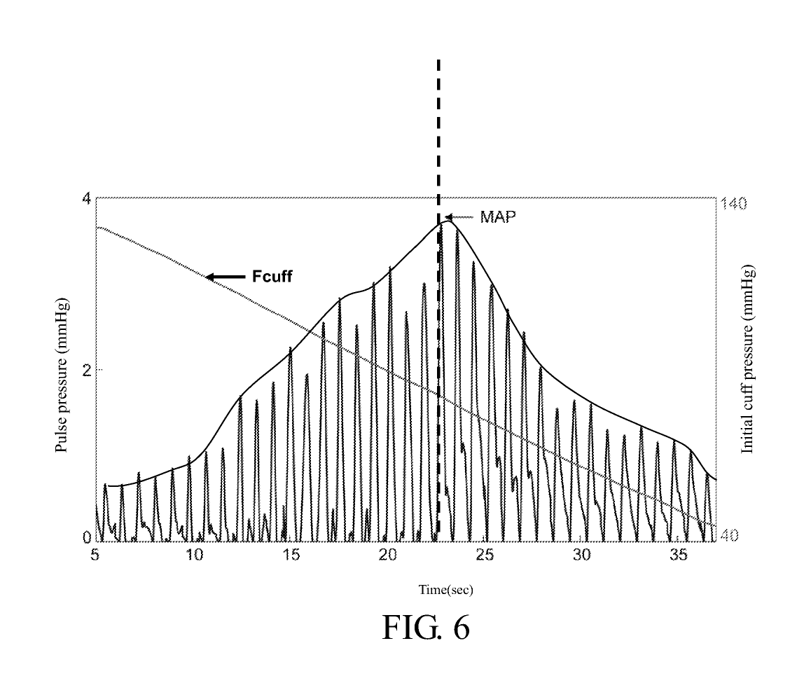

Please refer to FIG. 6, which is a diagram showing an oscillating waveform of the present disclosure. In FIG. 6, Fₑᵥ(t) is a curve profiling the characteristic of the envelope; F_{cuff}(t) is a curve profiling the characteristic of cuff pressure; Fₑᵥ(t₁) is the amplitude of BP waveform envelop happening at t₁ time; and according the systolic pressure and the diastolic pressure can be obtained as following:

(A) Systolic pressure:

Aₛ=Fₑᵥ(t₁)*Cᵣₛ;

wherein,
Aₛ is the pressure amplitude when the cuff pressure equal to the systolic pressure;
Cᵣₛ is characteristic ratio with respective to systolic pressure, and is defined to be varied adaptively in the following manner:

(1) if there is no personal information inputted, then

Cᵣₛ=aₛ−bₛ*max(F_{cuff});

(2) if there is an age information being inputted, then

Cᵣₛ=cₛ+dₛ/x−eₛ/x², x: age;

(3) if there is an information relating to height and weight being inputted, then

Cᵣₛ=fₛ−gₛ*y+hₛ*y²−iₛ*y³ y: weight/height²;

(4) if there is an information relating to medical history being inputted, then

Cᵣₛ is ranged between 0.3 and 0.8 that is dependent upon the patient groups;

Fₑᵥ(tₛ)=Aₛ, and tₛ<t₁;

tₛ is the time when the cuff pressure is equal to the systolic pressure;
SBP=F_{cuff}(tₛ), whereas SBP is the systolic pressure;

(B) Diastolic pressure:

A_{d}=Fₑᵥ(t₁)*C_{rd};

wherein,
A_{d} is the pressure amplitude when the cuff pressure equal to the diastolic pressure;
C_{rd} is characteristic ratio with respective to diastolic pressure, and is defined to be varied adaptively in the following manner:

(5) if there is no personal information inputted, then

C_{rd}=a_{d}−b_{d}*max(F_{cuff});

(6) if there is an age information being inputted, then

C_{rd}=c_{d}+d_{d}/x−e_{d}/x², x: age;

(7) if there is an information relating to height and weight being inputted, then

C_{rd}=f_{d}−g_{d}*y+h_{d}*y²−i_{d}*y³; y: weight/height²

(8) if there is an information relating to medical history being inputted, then

C_{rd} is ranged between 0.35 and 0.85 that is dependent upon the patient groups;

Fₑᵥ(t_{d})=A_{d}, and t_{d}>t₁;

t_{d} is the time when the cuff pressure is equal to the diastolic pressure;
DBP=F_{cuff}(t_{d}), whereas DBP is the diastolic pressure.

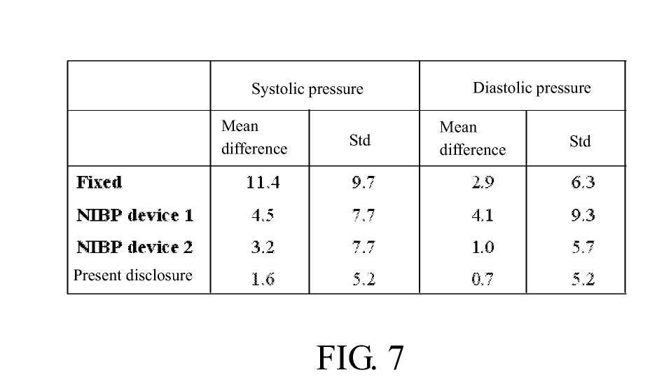

Please refer to FIG. 7, which is a table showing the comparison between the blood pressure measuring device of the present disclosure and other conventional blood pressure measuring devices. For comparison, a test procedure conforming to the EN1060-4 BP regulation is performed using a common non-invasive blood pressure simulation system, i.e. a NIBP simulator, according to that a cuff 6 mounted on a dummy arm is connected respectively to an non-invasive automatic blood pressure monitor and the NIBP simulator by the use of a T tube for simulating a condition of blood pressure measurement. During the process, the NIBP simulator will repeatedly play the oscillometric waveforms of heart beat and blood pressure that are recorded therein, and the same time monitor the blood pressure value measured by the automatic blood pressure monitor for determining the difference between the blood pressure value of the automatic blood pressure monitor and the reference value registered in the records. As illustrated in the table shown in FIG. 7, after comparing the performance of a common blood pressure monitor using fixed coefficient method, two NIBP devices and a blood pressure measuring device 1 of the present disclosure using 255 records registered in the NIBP simulator that are conforming to EN1060-4, it is obvious that the mean difference as well as the standard deviation of the present disclosure with respect to both the systolic and diastolic pressures are the smallest comparing with those of the other conventional devices. Thus, it can be concluded that the device and method for blood pressure measurement of the present disclosure are capable of achieving an accurate blood pressure measurement without being affected by the.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. A device for blood pressure measurement, comprising: a display unit, for displaying options to be selected by a user; an input unit, provided for the user to selectively input a personal health information or to proceed directly to perform a blood pressure measuring process; an inflation unit; a microprocessor; a cuff; an exhaust valve; a pressure sensor; a signal processing unit; and a data processing unit; wherein, the microprocessor is enabled to issue a command for directing the inflation unit to inflate the cuff to a specific pressure, and then the inflation of the cuff is stopped for allowing the cuff to be exhausted by releasing air from the deflation valve; and during the exhausting of the cuff, the pressure sensor is enabled to register a pressure signal while feeding the pressure signal to the signal processing unit where it is processed into a data conforming to a format recognizable and usable by posterior calculation processes.

2. A method for blood pressure measurement, comprising the steps of:
enabling a signal processing unit to perform a signal process operation and thus generating a signal waveform;
enabling a data processing unit to capture a mean arterial pressure (MAP) information from the signal waveform;
selecting an operation mode from the group consisting of a first mode and a second mode by a user to be used for selecting an adaptive characteristic coefficient corresponding to the selected operation mode; and
enabling a data processing unit to work in conjunction with a microprocessor for multiplying the adaptive characteristic coefficient with a mean arterial pressure value so as to measure and obtain a systolic pressure and a diastolic pressure while displaying the two on a display unit.

3. The method of claim 2, wherein the first mode is an automatic adaptive (a-adaptive) mode for defining the adaptive characteristic coefficient to be selected according to an initial cuff pressure of a cuff mounted on the user so as to be used for the measuring and determination of the systolic pressure and the diastolic pressure.

4. The method of claim 2, wherein the second mode is an intelligent adaptive (i-adaptive) mode for defining the adaptive characteristic coefficient to be selected according to a personal information inputted by the user including the age, height, and weight, or a physical characteristic selected from the group consisting of: the user's age, the user's height and the user's weight, so as to be used for the measuring and the determination of the systolic pressure and the diastolic pressure.

5. The method of claim 4, wherein the inputted personal information of the user further includes the medical history of the user, and the medical history further includes healthy group and patient groups, and the medical history in the second mode is provided as one factor for selecting the adaptive characteristic coefficient so as to determine the systolic pressure and the diastolic pressure.

6. A device for blood pressure determination, comprising: a display unit, for displaying options to be selected by a user; an input unit, provided for the user to selectively input a personal health information or to proceed directly to perform a blood pressure measuring process; an inflation unit; a microprocessor; a cuff; an deflation valve; a pressure sensor; a signal processing unit; a calculation unit; and a data processing unit; wherein, the microprocessor is enabled to issue a command for directing the inflation unit to inflate the cuff to a specific pressure, and then the inflation of the cuff is stopped for allowing the cuff to be exhausted by releasing air from the deflation valve; and during the exhausting of the cuff, the pressure sensor is enabled to register a pressure signal while feeding the pressure signal to the signal processing unit where it is processed into a data conforming to a specific format recognizable and usable by posterior calculation processes, and thereafter, transmitting the data with the specific format to the microprocessor where it is used in conjunction with an adaptive characteristic coefficient that is suitable for the user and is determined by the data processing unit for obtaining a systolic pressure and a diastolic pressure of the user while displaying the two on the display unit.
